(19) United States
(12) Patent Application Publication
(10) Pub. No.: US 2025/0186145 A1
(43) Pub. Date: Jun. 12, 2025

(54) Operating Room Including Autonomous Vehicles

(71) Applicant: NEOENTA LLC, Boston, MA (US)

(72) Inventor: Namal NAWANA, Weston, MA (US)

(73) Assignee: Neoenta LLC, Boston, MA (US)

(21) Appl. No.: 18/867,738

(22) PCT Filed: Jun. 6, 2023
(86) PCT No.: PCT/US2023/024587

§ 371 Date: Nov. 20, 2024

Related U.S. Application Data
(60) Provisional application No. 63/350,057, filed on Jun. 8, 2022.

(51) Int. Cl. A61B 34/32; A61B 90/00; G05D 1/667; G05D 105/30; G05D 109/25

(52) U.S. Cl. CPC A61B 34/32; A61B 90/361; G05D 1/667; G05D 2105/30; G05D 2109/254

(57) ABSTRACT

A system includes a robotic surgical system and an autonomous vehicle. The robotic surgical system includes a surgical tool. The autonomous vehicle is configured to remove the surgical tool from the robotic surgical system.

### RELATED APPLICATIONS

This application claims priority to U.S. provisional application No. 63/350,057 filed on Jun. 8, 2022, entitled “Operating Room Including Autonomous Vehicles,” which is incorporated herein by reference in its entirety.

### TECHNICAL FIELD

The following relates to an operating room and more particularly to an operating room including autonomous vehicles and associated methods for performing surgical procedures.

BACKGROUND

An operating room (“OR”) or operation suite is a sterile facility wherein surgical procedures are carried out. Generally, an OR includes a patient table, an overhead light, an anesthesia machine, and surgical instruments. Some ORs may further include one or more medical imaging systems that provide a real-time medical image of an anatomical feature (e.g., an organ) of a patient and a robotic surgical system that aids a surgeon in performing a surgical procedure.

Unfortunately, medical imaging systems, robotic surgical systems and other equipment needed to perform a surgical procedure typically occupy a large spatial volume including a great deal of floor space. As a result, hospitals desiring to include operating rooms with such systems must renovate existing spaces or build additional facilities large enough to accommodate the necessary equipment. The renovations or additions to the hospital are costly and may reduce a total number of operating rooms within a hospital as multiple operating rooms may be combined during a renovation.

### SUMMARY

Aspects of the present disclosure address the above-referenced problems and/or others.

In one aspect, a system includes a robotic system and an autonomous vehicle (AV) that can interact with one another to facilitate the performance of a surgical procedure. For example, the AV can interact with the robotic system to provide the needed surgical tools to the robotic system. By way of example, in some such embodiments, the AV can be configured to provide a surgical tool to the robotic system and remove from the robotic system a previously supplied surgical tool. Alternatively, the AV, or the robotic system itself, can store the previously supplied surgical tools on the robotic system. For example, in one case, the robotic surgical system includes a surgical tool. The autonomous vehicle is configured to remove the surgical tool from the robotic surgical system and to attach a second surgical tool to the robotic surgical system.

In some embodiments, the autonomous vehicle and the robotic surgical system can connect to a metaverse. In some embodiments, the robotic surgical system can include a first robotic arm with a first surgical tool removably attached thereto and a second robotic arm with a second surgical tool removably attached thereto. In these embodiments, the autonomous vehicle can remove the first and second tools and attach a third tool to a robotic arm.

In some embodiments, when the robotic surgical system and the autonomous vehicle are connected to a metaverse, the metaverse can include information (or can be provided with information) regarding a real time position of the autonomous vehicle. When the autonomous vehicle includes an optical camera, the metaverse can receive a real time video provided by the optical camera. In some embodiments, the autonomous vehicle can be a drone that is configured to automatically remove a surgical tool. In some embodiments, the robotic surgical system can be an autonomous vehicle.

In some embodiments, the system further includes a metaverse and a user computer system. The user computer system and the autonomous vehicle can connect to the metaverse and the user computer system can be configured to pilot the autonomous vehicle. In other embodiments, the system further includes a metaverse and a medical imaging system configured to provide a medical image (e.g., an image of an anatomical feature, e.g., an external or internal organ) of a subject and output the image to the metaverse. In some embodiments, the output image can be a real time image.

In another aspect, a first autonomous vehicle and a second autonomous vehicle can be configured to provide a medical image of a subject (e.g., the image of an anatomical feature (e.g., an external or an internal organ) of a subject and can be further configured to connect to a metaverse. In some embodiments, the first autonomous vehicle includes a radiation source that is configured to emit radiation that is attenuated by the subject and the second autonomous vehicle includes a radiation detector configured to detect the attenuated radiation. The first autonomous vehicle and the second autonomous vehicle can be configured to automatically image the subject. In some embodiments. metaverse includes a real time position of the first autonomous vehicle and the second autonomous vehicle. In some embodiments, the first autonomous vehicle and the second autonomous vehicle are drones.

By way of example, in some embodiments, a plurality of autonomous vehicles can be configured to cooperatively provide an imaging system. For example, one autonomous vehicle (e.g., a drone) can carry an X-ray emission source and another autonomous vehicle (e.g., a drone) can carry an X-ray sensor for detecting X-ray radiation. The X-ray emitting autonomous vehicle can be positioned relative to an anatomical feature for which an X-ray image is needed, e.g., relative to a portion of a patient's arm, and the X-ray detecting autonomous vehicle can be positioned relative to that anatomical feature to detect X-ray radiation passing through that feature so as to generate an X-ray image of the anatomical feature. The detection signals generated by the X-ray detecting autonomous vehicle can be analyzed by an analyzer residing on that autonomous vehicle or residing on a console in the operating room that is in communication with the autonomous vehicle.

In yet another aspect, a system for performing a surgical procedure includes a first autonomous vehicle configured to carry a tent and a second autonomous vehicle configured to sterilize an interior of the tent. In some embodiments, the first and second autonomous vehicles are drones. In some embodiments, the second autonomous vehicle includes an aerosol spray canister for sanitizing the interior of the tent. In some embodiments, the second autonomous vehicle includes a light source for sanitizing the interior of the tent. In some embodiments, the first autonomous vehicle is configured to carry the tent in an undeployed state and is further configured to release the tent and the tent includes a pump configured to place the tent in a deployed state when released. In some embodiments, the system further includes a robotic surgical system. In some embodiments, the robotic surgical system is an autonomous vehicle. In some embodiments, the system further comprises an aesthesia machine, wherein the anesthesia machine is an autonomous vehicle.

In yet another aspects, a system for performing a surgical procedure in an operating room (OR) includes at least a first autonomous vehicle (AV) configured for delivery of one or more surgical tools for performing said surgical procedure to the OR, at least a second AV coupled to an imaging system for acquiring one or more medical images of a patient, and at least one controller operably coupled to said first and second AV for controlling operation thereof. In some embodiments, the controller is configured to transmit one or more command signals to said first AV to instruct the AV to collect said one or more surgical tools from a repository of surgical tools and to deliver said collected surgical tools to said OR. In some embodiments, the controller is configured to transmit one or more command signals to said second AV to instruct the second AV to acquire said one or more medical images. In some embodiments, one or more medical images comprise X-ray images. In some embodiments, command signals instruct the second AV to acquire said one or more medical images of the patient during at least one of the following temporal intervals: (1) prior to commencement of the surgical procedure; (2) during performance of the surgical procedure; and (3) subsequent to completion of the surgical procedure. In some embodiments, the system further includes one or more robots for assisting performance of said surgical procedure. In some embodiments, the controller is configured to control operation of said one or more robots. In some embodiments, the controller is configured to coordinate interaction of at least one of said Avs with said one or more robots.

### BRIEF DESCRIPTION OF THE DRAWINGS

Aspects of the present disclosure may take form in various components and arrangements of components, and in various steps and arrangements of steps. The drawings are only for illustration purpose of preferred embodiments of the present disclosure and are not to be considered as limiting.

Features of embodiments of the present disclosure will be more readily understood from the following detailed description take in conjunction with the accompanying drawings in which:

FIG. 1 schematically depicts a computer system in accordance with an exemplary embodiment;

FIG. 2 schematically depicts a cloud computing environment in accordance with an exemplary embodiment;

FIG. 3 schematically depicts a metaverse network in accordance with an exemplary embodiment;

FIG. 4 schematically depicts an autonomous vehicle in accordance with an exemplary embodiment;

FIG. 5 illustrates a drone in accordance with an exemplary embodiment;

FIG. 6 depicts an operating room in accordance with an exemplary embodiment;

FIG. 7 depicts an anesthetic machine in accordance with an exemplary embodiment;

FIG. 8 depicts a robotic surgical system in accordance with an exemplary embodiment;

FIG. 9 depicts a medical imaging system in accordance with an exemplary embodiment;

FIG. 10 depicts an autonomous vehicle (e.g., a drone) that is configured to remove a surgical tool form a medical imaging system in accordance with an exemplary embodiment;

FIG. 11 depicts a drone carrying a tent in accordance with an exemplary embodiment;

FIG. 12 depicts a tent in a deployed state in accordance with an exemplary embodiment;

FIG. 13 depicts a drone with an aerosol spray canister for sanitizing an environment in accordance with an exemplary embodiment;

FIG. 14 depicts a drone with a sanitizing light in accordance with an exemplary embodiment;

FIG. 15 depicts a mobile imaging system in accordance with an exemplary embodiment;

FIG. 16 depicts a path of autonomous vehicles (e.g., drones) of a mobile imaging system in accordance with an exemplary embodiment; and

FIG. 17 depicts an optometric robot in accordance with an exemplary embodiment.

### DETAILED DESCRIPTION

A computer system or device as used herein, includes any system/device capable of receiving, processing, and/or sending data. Examples of computer systems include, but are not limited to personal computers, servers, hand-held computing devices, tablets, smart phones, multiprocessor-based systems, mainframe computer systems, and distributed cloud computing environments that include any of the above systems and the like.

The term “operating room” is used broadly to include any sterile environment, e.g., any sterile enclosure, in which surgical procedures can be performed. For example, an operating room can be a sterile room in a conventional building in which surgical procedures can be performed. As another example, an operating room may be tent providing a sterile enclosure in which surgical procedures can be performed. As discussed in more detail below, such a tent can store in a undeployed configuration and be deployed when needed to provide a sterile environment for performing surgical procedures.

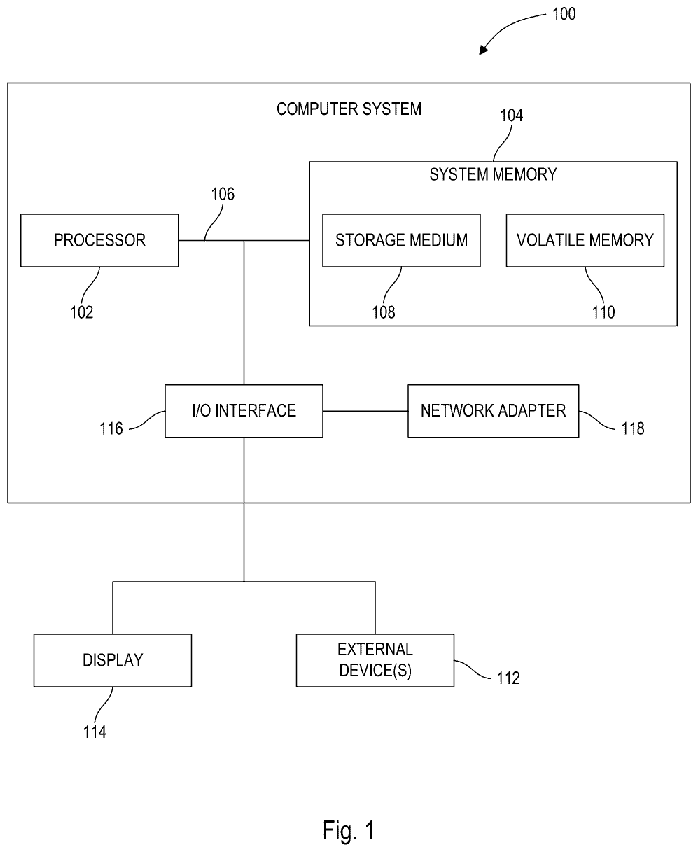

FIG. 1 depicts an exemplary computer system 100. The computer system 100 includes one or more processors or processing units 102, a system memory 104, and a bus 106 that couples various components of the computer system 100 including the system memory 104 to the processor 102.

The system memory 104 includes a computer readable storage medium 108 and volatile memory 110 (e.g., Random Access Memory, cache, etc.). As used herein, a computer readable storage medium includes any media that is capable of storing computer readable program instructions and is accessible by a computer system. The computer readable storage medium 108 includes non-volatile and non-transitory storage media (e.g., flash memory, read only memory (ROM), hard disk drives, etc.). Computer readable program instructions as described herein include program modules (e.g., routines, programs, objects, components, logic, data structures, etc.) that are executable by a processor. Furthermore, computer readable program instructions, when executed by a processor, can direct a computer system (e.g., the computer system 100) to function in a particular manner such that a computer readable storage medium (e.g., the computer readable storage medium 108) comprises an article of manufacture. Specifically, the execution of the computer readable program instructions stored in the computer readable storage medium 108 by the processor 102 creates means for implementing functions specified in methods disclosed herein.

The bus 106 may be one or more of any type of bus structure capable of transmitting data between components of the computer system 100 (e.g., a memory bus, a memory controller, a peripheral bus, an accelerated graphics port, etc.).

The computer system 100 may include one or more input devices 112 and a display 114. As used herein, an external device includes any device that allows a user to interact with a computer system (e.g., mouse, keyboard, touch screen, etc.). An input device 112 and the display 114 can be in communication with the processor 102 and the system memory 104 via an Input/Output (I/O) interface 116.

The display 114 may provide a graphical user interface (GUI) that may include a plurality of selectable icons and/or editable fields. A user may use an input device 112 (e.g., a mouse) to select one or more icons and/or edit one or more editable fields. Selecting an icon and/or editing a field may cause the processor 102 to execute computer readable program instructions stored in the computer readable storage medium 108. In one example, a user may use an input device 112 to interact with the computer system 100 and cause the processor 102 to execute computer readable program instructions relating to methods disclosed herein.

The computer system 100 may further include a network adapter 118 which allows the computer system 100 to communicate with one or more other computer systems/devices via one or more networks (e.g., a local area network (LAN), a wide area network (WAN), a public network (the Internet), etc.).

The computer system 100 may serve as various computer systems discussed throughout the disclosure.

A “cloud computing environment” provides access to shared computer resources (e.g., storage, memory, applications, virtual machines, etc.) to one or more computer systems.

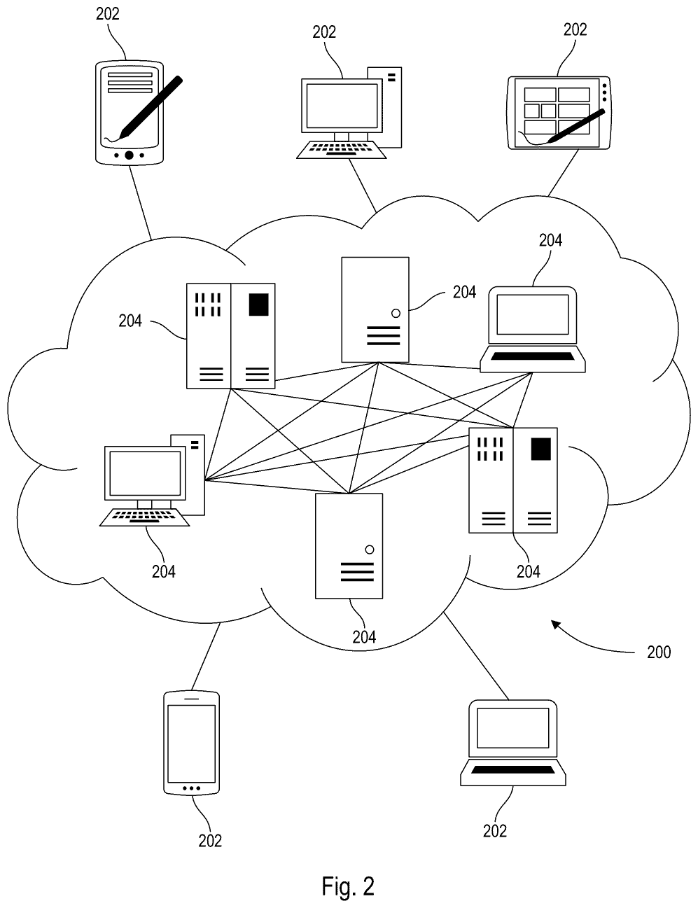

FIG. 2 depicts an exemplary cloud computing environment 200. The cloud computing environment 200 provides network access to shared computing resources (e.g., storage, memory, applications, virtual machines, etc.) to the one or more user computer systems 202 (e.g., a computer system 100) that are connected to the cloud computing environment 200. As depicted in FIG. 2, the cloud computing environment 200 includes one or more interconnected nodes 204. Each node may be a computer system or device with local processing and storage capabilities. The nodes 204 may be grouped and in communication with one another via one or more networks. This allows the cloud computing environment 200 to offer software services to the one or more user computer systems 202 and as such, a user computer system 202 does not need to maintain resources locally.

In one embodiment, a node 204 includes a system memory with computer readable program instructions for carrying out steps of the various methods discussed herein. In this embodiment, a user of a user computer system 202 that is connected to the cloud computing environment 200 may cause a node 204 to execute the computer readable program instructions stored in a node 204.

The cloud computing environment 200 may serve as various cloud computing environments discussed throughout the disclosure.

A “metaverse” as used herein refers to a virtual reality environment provided by one or more computer systems. A “metaverse network” refers to a network that allows_a user of a computer system to interact with a metaverse.

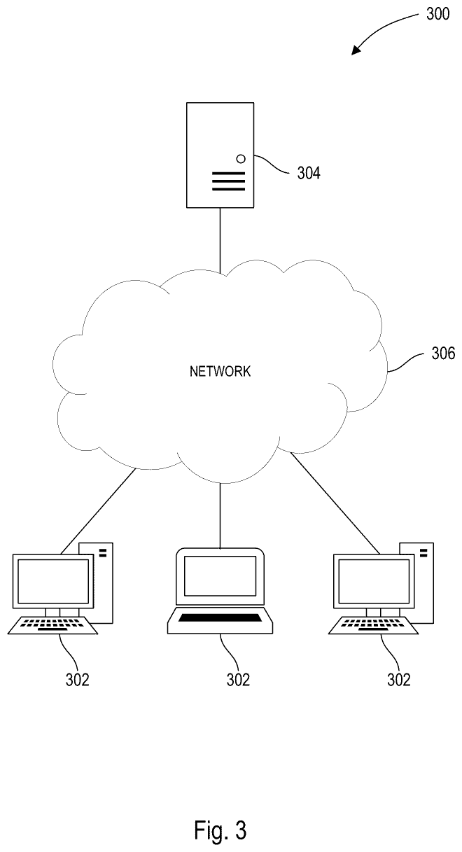

Referring now to FIG. 3, a metaverse network 300 is shown in accordance with an exemplary embodiment. The metaverse network 300 includes a plurality of user computer systems 302, a metaverse server 304, and a network 306. While FIG. 3 depicts the metaverse network 300 as including three user computer systems 302 and one metaverse sever 304, in other embodiments the metaverse network 300 may include more or less user computer systems 302 (e.g., 2, 5, 7, etc.) and more than one metaverse server 304 (e.g., 2, 3, 6, etc.). The user computer systems 302 are connected to and interface with the metaverse server 304 via a network (e.g., a local area network (LAN), a wide area network (WAN), a public network (the Internet), etc.).

The metaverse server 304 hosts a metaverse with which the users of a computer system 302 may interact. In one embodiment, a specified area of the metaverse is simulated by a single server instance and the metaverse server 304 may include a plurality of instances. The metaverse server 304 may also include a plurality of physics servers configured to simulate and manage interactions, collisions, etc. between characters and objects within the metaverse. The metaverse server 304 may further include a plurality of storage servers configured to store data relating to characters, media, objects, related computer readable program instructions, etc. for use in the metaverse.

The network 306 may employ traditional internet protocols to allow communication between user computer systems 302 and the metaverse server 304. In some embodiments, the user computer systems 302 may be directly connected to the metaverse server 304.

A user computer system 302 includes a metaverse client and a network client saved within a storage medium. In other embodiments the metaverse client and the network client may be stored in a different location that is accessible to a processor of the user computer system 302 (e.g., in a storage medium of a cloud computing environment). The metaverse client and the network client include computer readable program instructions that may be executed by a processor of the user computer system 302. When executed, the metaverse client allows a user of a computer system 302 to connect to the metaverse server 304 via the network 306 thereby allowing a user of the user computer system 302 to interact with the metaverse provided by the metaverse server 304. The metaverse client further allows a user of a user computer system 302 to interact with other users of other computer systems 302 that are also connected to the metaverse server 304. A user computer system 302 that is connected to the metaverse server 304 may be said to be connected to a metaverse. Accordingly, a user computer system 302 is configured to connect to a metaverse.

The network client, when executed by a processor, facilities connection between the user computer system 302 and the metaverse server 304 (i.e., by verifying credentials provided by the user). For example, when executed and a user of a computer system 302 requests to log onto the metaverse server 304, the network client maintains a stable connection between the user computer system 302 and the metaverse server 304 and handles commands input by a user of a computer system 302 and handles communications from the metaverse server 304.

When a user of the user computer system 302 is logged into the metaverse server 304, a display connected to the computer system 302 conveys a visual representation of a metaverse provided by the metaverse server 304.

The metaverse serve 304 may provide various metaverses discussed throughout the disclosure.

As used herein a “virtual reality headset” or VR headset refers to a head mounted display system with left and right displays that allow a user to view an image (or video) in a lifelike environment. The VR headset includes a computer system or is connected to an external computer system via a wired or wireless connection. This computer system process images and outputs the images to the left and right displays of the VR headset such that a user may view the images in a lifelike environment. For example, a stereoscopic camera may capture an image that is appropriately shown in the left and right displays of the VR headset. A VR headset also includes a tracking system that tracks a user's head orientation and position. Such a tracking system may include accelerometers, gyroscopes, magnetometers, motion processors, infrared tacking, and other devices capable of tracking a head position. The tracking system sends a signal indicative of head position to the connected computer system and in response, the computer system updates the output image such that image is adjusted based on the user's head movement.

In some embodiments, the computer system 302 may be connected to a VR headset. In these embodiments, the metaverse server 304 provides a metaverse to the displays of the VR headset thereby creating a lifelike environment for the user.

In other embodiments, an adjustable stereoscopic camera provides a live video feed to a connected VR headset. In these embodiments, the position of the stereoscopic camera may be based on a user's head movement such that the provided video is adjusted based on where the user is looking.

A “vehicle” as used herein refers to a machine that transports cargo from one location to another. A vehicle includes a drive system (e.g., a motor, drivetrain, wheels, propellor, etc.). An “autonomous vehicle” (“AV”) as used herein refers vehicle with self-piloting elements.

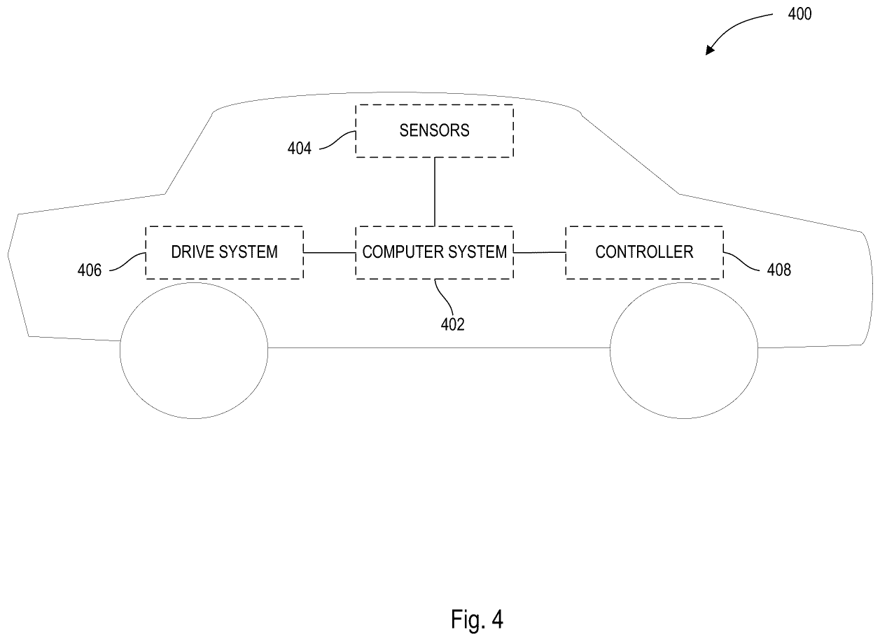

FIG. 4 depicts an exemplary autonomous vehicle 400. While FIG. 4 depicts the autonomous vehicle as a car, the autonomous vehicle 400 may be another type of vehicle (e.g., a drone). The AV 400 includes a computer system 402 that is connected to and in communication with a plurality of sensors 404 (e.g., radar, lidar, sonar, GPS, optical cameras, thermographic cameras, etc.) and a drive system 406 (e.g., a motor, drivetrain, wheels, etc.) that is also connected to and in communication with the computer system 402. The computer system 402 receives a destination (e.g., from a user input) and in response to receiving the destination causes the drive system 406 to move the AV 400 to the indicated destination. While moving, the computer system 402 may receive from the sensors 404 one or more signals indicative of one or more obstacles in the path of the AV 400. In response to receiving these signals, the computer system 402 causes the drive system 406 to adjust a path of the AV 400 in order to avoid the obstacle(s). Together, the computer system 402, the sensors 404, and the drive system 406 pilot an autonomous vehicle from one location to another. In some embodiments, the AV 400 includes a controller 408 that is connected to and in communication with the computer system 402. In some embodiments, the controller 408 may be external from the AV 400. The controller 408 may override the self-piloting features of the AV 400 and allow a user to remotely pilot the AV 400. Stated another way, the controller 408 may send a control signal to the computer system 402 based on a user input. The computer system 402 causes the drive system 406 to move the AV 400 based on the control signal.

The autonomous vehicle 400 may serve as various autonomous vehicles discussed throughout the disclosure.

A “drone” as used herein refers to an unmanned aerial vehicle. A drone can be an autonomous vehicle or may be piloted remotely by a human pilot.

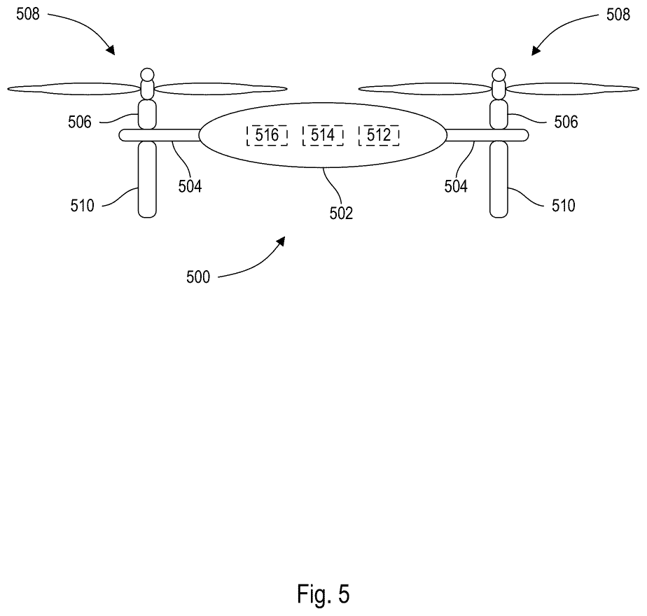

FIG. 5 depicts an exemplary drone 500. The drone 500 includes a body 502, arms 504, motors 506, propellers 508, and landing legs 510. The proximal ends of the arms 504 are connected to the body 502 and distal ends of the arms 504 are connected to the motors 506 and the landing legs 510. The motors 506 are connected to and drive the propellers 508 and the landing legs 510 support the drone 500 during takeoff and landing.

The body 502 houses a battery 512 that powers the drone 500 and a computer system 514. The computer system 514 is connected to and in communication with the motors 506, a plurality of sensors 516 (e.g., radar, lidar, sonar, GPS, optical cameras, thermographic cameras, etc.) disposed within the body 502 or on a surface of the body 502, and an external computer system (e.g., controller, tablet, smartphone, personal computer, etc.). The computer system 514 causes the motors 506 to drive the propellers 508 at various rotation rates in order to properly maneuver the drone 500. The computer system 514 causes the drone 500 to move based on signals from the external computer system (e.g., a signal indicative of an input destination).

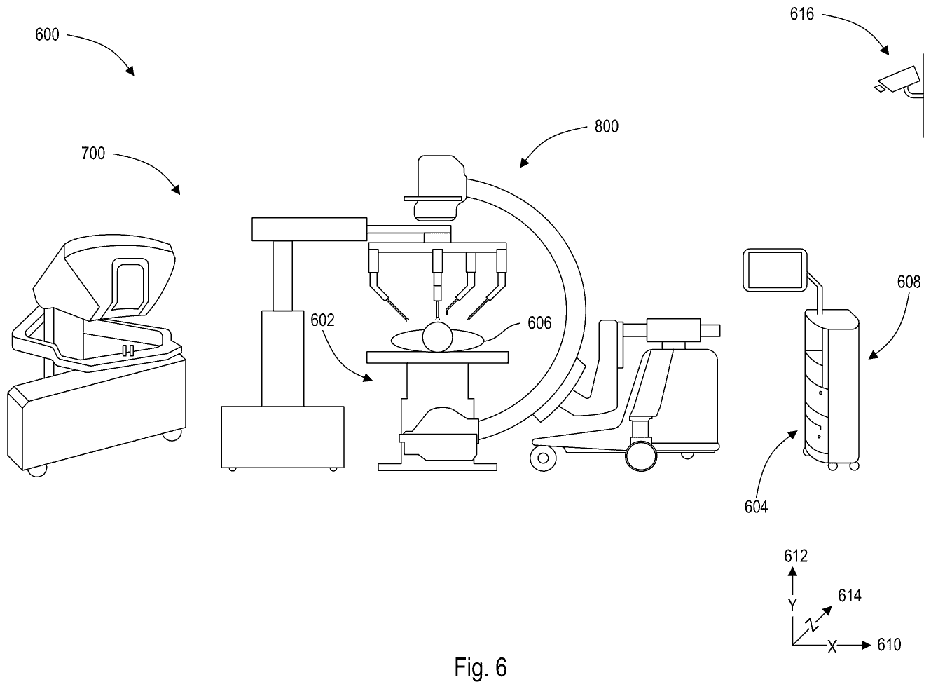

Referring now to FIG. 6, an operating room 600 is shown in accordance with an exemplary embodiment. In this embodiment, the operating room 600 includes a patient table 602, a computer system 604, an anesthesia machine 700, a robotic surgical system 800, and a medical imaging system 900.

The patient table 602 supports a patient 606 that is undergoing a surgical procedure. The patient table 602 may move vertically and horizontally in order to properly position the patient 606. While the patient table 602 is depicted as a stationary table, in some embodiments the patient table 602 is movable, e.g., via application of control signals thereto or by a human operator.

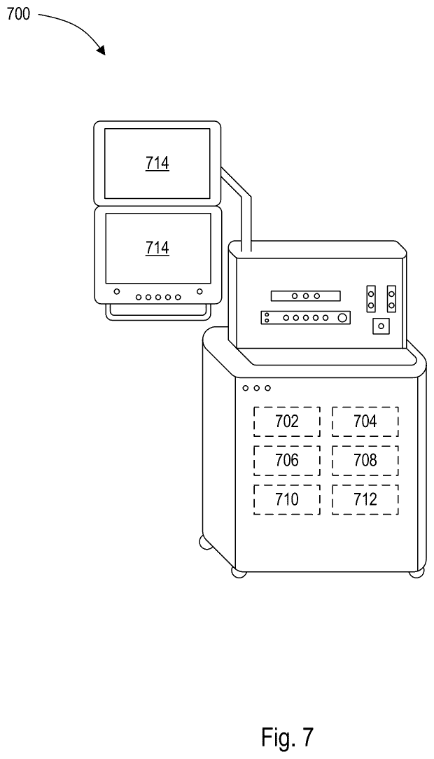

While not depicted in FIG. 6, the operating room 600 may further include an anesthesia machine 700 (FIG. 7) configured to anesthetize the patient 606. As used herein anesthetizing a patient can include generally anesthetizing a patient, regionally anesthetizing a patient, locally anesthetizing a patient, or sedating a patient.

In some embodiments, the anesthesia machine 700 is an AV and as such, the anesthesia machine 700 may include one or more sensors, a drive system, and a computer system needed to autonomously pilot the anesthesia machine 700. In these embodiments, the anesthesia machine 700 can move from a storage room to the operating room 600. The anesthesia machine 700 may automatically move to the operating room 600 based on a predetermined schedule (e.g., a surgery schedule) or based on a user input. When a surgical procedure is complete and the anesthesia machine 700 is no longer needed, the anesthesia machine 700 may automatically return to the storage room and may be automatically connected to docking elements disposed therein.

In this embodiment, the anesthesia machine 700 includes a vaporizer 702 configured to supply an anesthetic agent to a subject. More particularly, the vaporizer 702 includes a reservoir that contains an anesthetic agent that is to be delivered to a patient. The vaporizer 702 may be removed from the anesthesia machine 700 and replaced with a different vaporizer 702 with a different anesthetic agent. The reservoir includes a lower portion that contains the anesthetic agent in a liquid form and an upper portion that contains the anesthetic agent in a vaporized form. During operation, a combination of temperature and pressure cause the liquid anesthetic agent to vaporize and enter the upper portion of the reservoir.

The anesthesia machine 700 further includes one or more tanks 706 that hold various gases (e.g., oxygen, nitrous oxide, etc.). The tank(s) 706 are connected to the reservoir via one or more conduits. Gas provided by the tanks 706 enters the reservoir of the vaporizer 702 and mixes with the vaporized anesthetic agent to form breathing gas.

The anesthesia machine 700 further includes a ventilator 704 that is connected to and in communication with the vaporizer 702. The ventilator 704 is configured to supply the breathing gas to the patient 606 via a breathing circuit (not shown). In these embodiments, the breathing circuit may be coupled between an airway of the patient 606 (e.g., via a breathing mask positioned over the nose and/or mouth of the patient 606) and the ventilator 704. Accordingly, breathing gases flow from the ventilator 704 and into the airway of the patient 606 via the breathing circuit.

The anesthesia machine 700 also includes flow rate adjuster 706 that is configured to adjust an amount of anesthetic agent delivered to the patient 606. The flow rate adjuster 708 changes an amount of agent delivered to the patient 606 by adjusting the flow rate of the gases from the one or more tanks 706. The flow rate adjuster 708 includes one or more analog or digital adjustment devices that allow an operator (e.g., an anesthesiologist) to adjust the flow rate. For example, the anesthesia machine 700 may include one or more adjustable valves positioned between the vaporizer 702 and the connected gas tanks 706. An operator may adjust a position of a valve via an adjustment device thereby changing a flow rate of a gas. The anesthesia machine 700 may also include one or more bypass valves which allows a first portion of the gas from the gas tanks 706 to flow directly to the ventilator 704 and allows a second portion of the gas from the gas tanks 706 to flow to the vaporizer. The bypass valve allows an operator to control a concentration of vaporized anesthetic agent delivered to the patient 606 by adjusting the ratio of gas from the gas tank 706 to anesthetic agent from the vaporizer 702.

The anesthesia machine 700 further includes a respiratory gas module 710 and a computer system 712 that is connected to and in communication with the respiratory gas module 710. The respiratory gas module 710 is configured to measure various parameters of gases exiting the vaporizer 702 and/or provided to the patient 606 via the ventilator 704. For example, the respiratory gas module 710 may measure concentrations of carbon dioxide, nitrous oxide, and anesthetic agent provided to the patient 606. The respiratory gas module 710 may also measure various patient parameters including, but not limited to, respiration rate, minimum alveolar concentration, and patient oxygen level.

The respiratory gas module outputs signals indicative of the measured parameters to the computer system 712. A processor of the computer system 712 processes the signals and outputs parameters indicative thereof to a display 714. An operator may view the parameters and may adjust a flow rate, concentration of anesthetic, etc. based on the parameters. In some embodiments, the computer system 712 may automatically adjust an amount/flow rate of anesthetic agent or other gas provided to the patient 606 based on the measured parameters.

The operator may control operating parameters of the anesthesia machine 700 via the computer system 712. For example, the operator may employ the computer system 712 to adjust flow rate of gases, concentration of anesthetic, etc. Based on these adjustments, the state of corresponding valves (e.g., open or closed or to what degree the valve is open or closed) within the anesthesia machine 700 may be changed accordingly. Particularly, the operator may employ the computer system 712 to increase or decrease flow of oxygen from a tank 706 to the patient 606.

The anesthesia machine 700 is described as an AV, which in some embodiments may be a drone. In such embodiments, a drone (e.g., the drone 500) carries or includes the components of the anesthesia machine 700.

A user of an external computer system that is connected to the computer system of the drone with the anesthesia machine 700 may input a target destination (e.g., coordinate position, room, etc.) which causes the external computer system to send one or more signals indicative of the input to the computer system of the drone.

In response to receiving such signal(s), the computer system of the drone causes the drone to decouple from the docking elements (e.g., a docking station). Since the drone is an AV, the drone can automatically travel to the target destination. In another embodiment, a user of the external computer system may manually pilot the drone to the target destination.

Upon arriving at the target destination, the drone positions/orients itself, e.g., based on previously received instructions or instructions received upon arrival at the target destination. In some embodiments, one or more optical camera(s) of the drone may automatically capture optical images of the target destination and send the image(s) to a computer system (e.g., the computer systems of the drones, nodes of a cloud computing system, etc.).

In response to receiving the image(s), the computer system may employ optical image recognition software that automatically identifies the target destination within the received optical images and sends position signals to the computer system of the drone indicative thereof. In response to receiving these signals, the computer system of the drone causes the drone to maneuver to a desired position. In other embodiments, a user of the external computer system pilots the drone to a position. Furthermore, in these embodiments a user may also adjust (set) the orientation of the drone (e.g., via setting the altitude and/or the azimuth angle).

Once in a proper position and orientation of the anesthesia drone is achieved, the anesthesia machine 700 may begin anesthetizing the patient. When a surgical procedure is complete, the drone may return to the storage room automatically or via a human pilot. In some embodiments, an anesthesiologist may view the procedure via a video captured by an optical camera of the drone. In these embodiments, the anesthesiologist may remotely control this drone and intervene (e.g., override actions taken by the drone) if needed.

As discussed with respect to the exemplary drone 500, in some embodiments a drone is connected to an external computer system and includes an optical camera. In one embodiment, the external computer system may be a user computer system that is connected to a metaverse server. Stated another way, a drone (or other autonomous vehicle) with an anesthesia machine 700 may be connected to a user computer system 302 that is connected to a metaverse server 306. In this embodiment, a metaverse server may generate a metaverse that depicts the drone with the anesthesia machine 700. The metaverse server may update a position/orientation of this drone within the metaverse as it moves to a target destination. Once the drone arrives at the target destination the metaverse server may populate a graphical representation of the target destination into the metaverse, may update a position of the drone, and may update a progress of the procedure. Once the procedure is complete, the metaverse server may update a position of the drone within the metaverse as it returns to a storage room. Furthermore, the metaverse server may populate a live video feed from the optical camera of the drone into the metaverse.

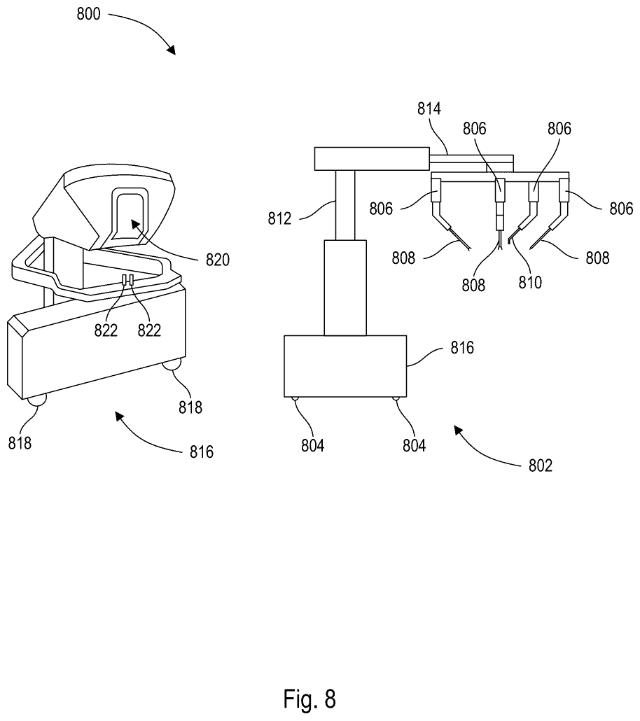

As depicted in FIG. 8, the robotic surgical system 800 includes a patient side cart 802. The patient side cart 802 can include wheels 804 that may be utilized to move the patient side cart 802. In some embodiments, the patient side cart 802 is an AV and as such, the patient side cart 802 may include one or more sensors, a drive system, and a computer system needed to autonomously pilot the patient side cart 802. In these embodiments, the patient side cart 802 may pilot itself from a storage room to the operating room 600. The patient side cart 802 may move to the operating room 600 based on a predetermined schedule (e.g., a surgery schedule) or based on a user input. When a surgical procedure is complete and the robotic surgical system 800 is no longer needed, the patient side cart 802 may automatically return to the storage room and may automatically connect to docking elements disposed therein.

The patient side cart 802 includes a plurality of robotic arms 806. Three of the robotic arms 806 are connected to a surgical tool 808 and a fourth robotic arm 806 is connected to a camera assembly 810. The robotic arms 806 are configured to move the surgical tools 808 and the camera assembly 810. The robotic arms 806 include robotic joints that allow the robotic arms 806 to move in various directions. The patient side cart 802 further includes drive elements (e.g., motors, servos, electromechanical actuators, etc.) that are configured to manipulate the surgical tools 808 and the camera assembly 810 once inside the patient. The surgical tools 808 may be inserted into the patient via a cannula. When inserted, a surgeon manipulates the surgical tools 808 to carry out a surgical procedure. The camera assembly 810 captures an image (e.g., live video image) of the surgical site and distal ends of the surgical tools 808 when the surgical tools 808 are within a field-of-view of the camera assembly 810. The camera assembly 810 may include, but is not limited to, a stereoscopic endoscope. The patient side cart 802 is connected to and in communication with the computer system 604 via a wired or wireless connection. As will be discussed in further detail herein, the camera assembly 810 outputs the captured image to the computer system 604 for further image processing.

As depicted in FIG. 6, the computer system 604 may be supported by a cart 608. In some embodiments, the cart 608 may be an AV and as such, the cart 608 may include one or more sensors and a drive system needed to autonomously pilot the cart 608. In these embodiments, the cart 608 may pilot itself from a storage room to the operating room 600. The cart 608 may move to the operating room 600 based on a predetermined schedule (e.g., a surgery schedule) or based on a user input. When a surgical and/or an imaging procedure is complete and the computer system 604 is no longer needed, the cart 608 may automatically return to the storage room and may automatically connect to docking elements disposed therein.

While the patient side cart 802 is depicted as supporting three surgical tools 808 and one camera assembly 810, in other embodiments the patient side cart 802 may support more or less surgical tools 808 and additional camera assemblies 810. The number of and/or type surgical tools 808 used at one time may depend on a surgical procedure being performed.

The surgical tools 808 may include, but are not limited to, scalpels, forceps, and catheters. The surgical tools 808 and the camera assembly 810 may be removably attached to the robotic arms 806. As such, first surgical tools 808 may be removed from the robotic arms 806 and be replaced with different second surgical tools 808. Such removable attachment may be achieved using, without limitation, a threaded attachment interface, a tongue and groove attachment interface, and/or a snap fit attachment interface. During some surgical procedures, it may be necessary to change surgical tools 808 during the surgical procedure. In these procedures, one or more surgical tools 808 may be removed from a robotic arm 806 and a different second surgical tool 808 may be coupled to the robotic arm 806.

The patient side cart 802 further includes a vertical support column 812 and a horizontal support column that are configured to align the robotic arms 806 (and therefore the surgical tools 808 and the camera assembly 810) with a surgical site. The robotic arms 806 are connected to the horizontal support column via a base 816. The vertical support column 812 is configured to move vertically and the horizontal support column 814 is configured to move horizontally and perpendicular to the vertical support column 812. Accordingly, the vertical support column 812 vertically moves the robotic arms 806 and the horizontal support column 814 horizontally moves the robotic arms 806.

While the patient side cart 802 is depicted as supporting the robotic arms 806, in other embodiments the patient side cart 802 may be omitted. In these embodiments the robotic arms 806 may be fixedly mounted within the operating room 600 (e.g., mounted to the ceiling or a wall of the operating room 600 or mounted to the patient table 602). When mounted to the ceiling or a wall of the operating room 600, the robotic arms 806 are moveable between a retracted and a deployed position. When in the deployed position, the robotic arms 806 align the surgical tools 808 and the camera assembly 810 with a surgical site.

The robotic surgical system 800 further includes a surgeon console 816. The surgeon console 816 includes wheels 818 that may be utilized to move the surgeon console 816. In some embodiments, the surgeon console 816 is an AV and as such, the surgeon console 816 may include one or more sensors, a drive system, and a computer system needed to autonomously pilot the surgeon console 816. In these embodiments, surgeon console 816 may pilot itself from a storage room to the operating room 600. The surgeon console 816 may move to the operating room 600 based on a predetermined schedule (e.g., a surgery schedule) or based on a user input. When a surgical procedure is complete and the surgeon console 816 is no longer needed, the surgeon console 816 may automatically return to the storage room and automatically connect to docking elements disposed therein.

While FIG. 6 depicts the surgeon console 816 as being disposed within the operating room 600 in other embodiments the surgeon console 816 may be remotely located relative to the operating room 600. Providing the surgeon console 816 in a different location than the operating room 600 may allow a surgeon to carry out a surgical procedure from a nonsterile location in which the surgeon console 816 is positioned.

The surgeon console 816 is connected to and in communication with the computer system 604 via a wired or wireless connection and includes a display 820, one or more control devices 822.

The computer system 604 receives the image captured by the camera assembly 810, a processor of the computer system 604 further processes the receives image, and outputs the processed image to the display 820 thereby allowing a surgeon to remotely view a surgical site. In some embodiments, the display 820 may be divided into a left eye display and a right eye display for providing a surgeon with a coordinated stereo view of the surgical site. In some embodiments, the display 820 may be within a VR headset.

In some embodiments, the computer system 604 includes or is connected to and in communication with a system memory that stores preoperative images/models (e.g., computer tomography (CT) image, magnetic resonance imaging system (MRI) image, ultrasound image, X-image, 3D MRI model etc.) that include a region of interest (e.g., including an anatomy to be operated on). In these embodiments, a surgeon may identify an anatomy of interest within the displayed image provided by the camera assembly 810 (e.g., by using an input device to manually label the anatomy of interest) or the computer system 604 may automatically determine the anatomy of interest. The location of the anatomy of interest may be correlated with a location of features within the stored preoperative images. In response to correlating the location, the computer system 604 may output a preoperative image with the anatomy of interest to the display 820 along with the image captured by the camera assembly 810. The computer system 604 may move the displayed preoperative image based on the relative location of the anatomy of interest in the displayed image captured by the camera assembly 810. For example, when the anatomy of interest moves to the left in the image captured by the camera assembly 810, the preoperative image shown by the display 820 is also shifted to the left.

When a stored 3D model includes the correlated anatomy of interest, computer system 604 may output the model and the image captured by the camera assembly 810 to the display 820. The orientation of the 3D model may be adjusted based on a surgeon input or may be automatically adjusted as the anatomy of interest moves within the image captured by the camera assembly 810.

In some embodiments, computer system 604 may further process images (i.e., the preoperative images and/or the images captured by the camera assembly 810) such that the displayed images include annotations, highlighting, bounding boxes, different contrast, etc. that provides information about or further highlights the anatomy of interest within the displayed preoperative image and/or the displayed 3D model. In further embodiments, the computer system 604 may further process the images to overlay at least a portion of the preoperative image or at least a portion of a stored 3D model onto the image captured by the camera assembly 810 using an image registration technique.

A surgeon manipulates the surgical tools 808 and the camera assembly 810 via the control devices 822 to carry out a surgical procedure. The surgeon may input a command (e.g., a command for moving a surgical tool) via a control device 822 which outputs a signal indicative of the input to the computer system 604. In response, the processor of the computer system causes the drive elements of the robotic arms 806 to move the surgical tools 808 and/or the camera assembly 810 based on the received signal. The input control devices 822 provide the same degrees of freedom, as the surgical tools 808 and the camera assembly 810. In some embodiments, the surgical tools 808 include position, force, and tactile feedback sensors that transmit position, force, and tactile sensations back to the control devices 822 via the computer system 604.

In some embodiments, the robotic arms 806 can mimic the movement of human arms and two robotic arms 806 (e.g., a left arm and a right arm) each correspond to a left and right arm of the surgeon. In these embodiments, a surgeon may wear a plurality of bands with arm tracking sensors (e.g., accelerometers, gyroscopes, magnetometers, motion processors, etc.) that are configured to determine a position and movement of the surgeon's arms. The arm tracking sensors are connected to and in communication with the computer system 604 via a wired or wireless connection. The arm tracking sensors send signals indicative of arm position to the computer system 604 and in response, the computer system 604 causes the corresponding robotic arms 806 to move in a similar manner.

Similarly, movement of the surgical tools can mimic finger movement or may be controllable with finger gestures. In these embodiments, the surgeon may also wear gloves with hand tracking sensors (e.g., accelerometers, gyroscopes, magnetometers, motion processors, etc.) that are configured to determine a position and movement of the surgeon's hands and fingers. The hand tracking sensors are connected to and in communication with the computer system 604 via a wired or wireless connection. The hand tracking sensors send signals indicative of hand and finger position to the computer system 604 and in response, the computer system 604 causes the corresponding surgical tools 808 to move.

The robotic surgical system 800 is described as an AV, which in some embodiments, may be a drone. In these embodiments, a drone (e.g., the drone 500) carries or includes the components of the elements of the patient side cart 802 needed to carry out a surgical procedure (e.g., articulable robotic arms 806, surgical tools 808, the camera assembly 810).

A user of an external computer system that is connected to the computer system of this drone may input a target destination (e.g., coordinate position, room, etc.) which causes the external computer system to send a signal indicative of the input to the computer system of the drone.

In response to receiving this signal, the computer system of the drone causes the drone to decouple from the docking elements (docking station). Since the drone is an AV, the drone can automatically travel to the target destination. In another embodiment, a user of the external computer system may manually pilot the drone to the target destination.

Upon arriving at the target destination, the drone positions and orients itself in accordance with instructions sent to the drone, e.g., via a remote controller. In some embodiments, an optical camera(s) of the drone may automatically capture optical images of the target destination and send the images to a computer system (e.g., the computer systems of the drones, nodes of a cloud computing system etc.). In response to receiving the images, the computer system may employ optical image recognition software that automatically identifies the target destination within the received optical images and sends position signals to the computer system of the drone indicative thereof. In response to receiving these signals, the computer system of the drone causes the drone to maneuver to a position/orientation. In other embodiments, a user of the external computer system pilots the drone to a position/orientation.

Once in a proper position/orientation, a surgeon may employ elements of the robotic surgical system 800 (e.g., the articulable robotic arms 806, surgical tools 808, and the camera assembly 810, the control devices 822, etc.) to carry out a surgical procedure. When a surgical procedure is complete, the drone may return to the storage room automatically or via a human pilot.

As discussed with respect to the exemplary drone 500, in some embodiments a drone is connected to an external computer system and includes an optical camera. In one embodiment, the external computer system may be a user computer system that is connected to a metaverse server. Stated another way, a drone (or other autonomous vehicle) with components of the robotic surgical system 800 may be connected to a user computer system 302 that is connected to a metaverse server 306. In this embodiment, a metaverse server may generate a metaverse that depicts the drone with the components of the robotic surgical system 800. The metaverse server may update a position/orientation of this drone within the metaverse as it moves to target destination. Once the drone arrives at the target destination the metaverse server may populate a graphical representation of the target destination into the metaverse, may update a position of the drone, and may update a progress of the procedure. Once the procedure is complete, the metaverse server may update a position of the drone within the metaverse as it returns to a storage room. Furthermore, the metaverse server may populate a live video feed from the optical camera of this drone into the metaverse.

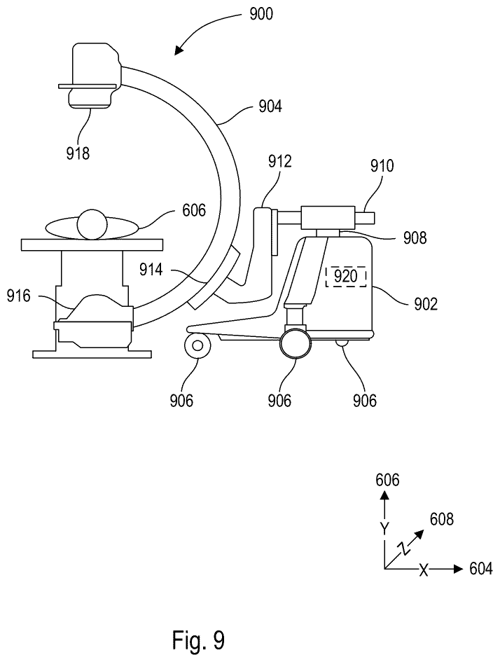

Certain surgical procedures may be aided by providing a real time view of an anatomical structure (e.g., internal anatomical structures, such as organs) of the patient 606. These procedures include but are not limited to minimally invasive catheter-based cardiac interventions (e.g., endovascular repair, cardiac ablation, aneurysm repair, etc.) and endoscopic transoral nasopharyngectomy (ETON). During these procedures, a medical imaging system 900 may acquire one or more images of an internal region of interest. As used herein, the medical imaging system 900 includes systems or devices that capture one or more images or videos of the patient 606. While FIGS. 6 and 9 depict the medical imaging system 900 as a C-arm X-ray imaging system, in other embodiments the medical imaging system 900 may be a different type of medical imaging system (e.g., a magnetic resonance imaging (MRI) system, a computed tomography system, a positron emission tomography (PET) system, an X-ray imaging system, an ultrasound system, etc.).

As depicted in FIG. 9, the medical imaging system 900 includes a cart 902 that supports a C-arm 904. The cart 902 includes wheels 906 that may be utilized to move the medical imaging system 900. In some embodiments, the medical imaging system 900 is an AV and as such, the medical imaging system 900 may include one or more sensors, a drive system, and a computer system needed to autonomously pilot the medical imaging system 900. In these embodiments, the medical imaging system 900 may pilot itself from a storage room to the operating room 400. The medical imaging system 900 may move to the operating room 400 based on a predetermined schedule (e.g., a surgery schedule) or based on a user input. When a surgical and/or an imaging procedure is complete and the medical imaging system 900 is no longer needed, the medical imaging system 900 may automatically return to the storage room and may automatically connect to docking elements disposed therein.

The medical imaging system 900 further includes a vertical support column 908 and a horizontal support column 910. The vertical support column 908 is configured to move vertically with respect to the cart 902. The horizontal support column 910 is configured to move horizontally and perpendicular to the vertical support column 908. Accordingly, the vertical support column 908 vertically moves the C-arm 904 and the horizontal support column 910 horizontally moves the C-arm 904. The medical imaging system 900 also includes a connection arm 912 and a rotation device 914. The connection arm 912 is connected to the horizontal support column 910 and the rotation device 914. The connection arm 912 is configured to pivot or rotate about an x-axis 610 of a standard Cartesian plane. The rotation device 914 is connected to the C-arm 904 and the connection arm 912. The rotation device 914 is configured to rotate around a z-axis 614 of a standard cartesian plane.

The C-arm 904 supports a radiation source (e.g., an X-ray tube) 916 and radiation detector 918 disposed at opposite ends of the C-arm 904. The radiation source 916 emits radiation that traverses an examination region and is attenuated by an object (e.g., the patient 606) that is within the examination region. The radiation detector 918 detects the attenuated radiation that has traversed the examination region and outputs a signal indicative thereof. A reconstructor reconstructs the output signals and generates image data that may be output to a display.

The rotational and horizontal and vertical movement of the C-arm 904 are/controlled by a drive system 920. The drive system 920 causes the horizontal support column 910, the vertical support column 912, the connection arm 912, and the rotation arm 914 to properly position/orient the radiation source 916 and the radiation detector 918 based on a user input or may automatically move the C-arm 904 to properly position/orient the radiation source 916 and the radiation detector 918 based on an imaging plan.

In some embodiments, the medical imaging system 900 is connected to and in communication with the computer system 604 via a wired or wireless connection In these embodiments, a user of the computer system 604 may input an instruction to start or stop radiation emission, may input a position/orientation of the C-arm 904 and/or may input an imaging plan at the computer system 604 and in response, the computer system 604 may cause radiation source to start or stop radiation emission and/or may the drive system 920 to move the C-arm 904 based a user input or based on the input imaging plan.

The computer system 604 is further connected to and in communication with the surgeon console 816. In some embodiments, the computer system 604 may include a reconstructor that generates image data and outputs an image on the display 820. In these embodiments, the computer system 604 may further process the image as previously discussed herein with respect to the computer system 604 processing an image captured by the camera assembly 810. Furthermore, when the display 820 is within a VR headset, the computer system 604 may properly output the image for viewing within a VR headset and may move the image based on a detected head movement as previously discussed herein.

In other embodiments, the imaging system 900 may be connected to a cloud computing environment (e.g., the cloud computing environment 200) and a node of a cloud computing environment may cause the radiation source to start or stop radiation emission and may cause the drive system 920 to move the C-arm 904 based on an imaging plan (e.g., an imaging plan stored in a node of a cloud computing environment or based on an imaging plan input at a user computer system connected to a cloud computing environment) or based on a user input (e.g., a user input imaging plan or a user input instruction to start or stop radiation emission and/or a user input C-arm 904 position/orientation) at a user computer system that is connected to the cloud computing environment. In these embodiments, the node of the cloud computing environment may include the reconstructor and may process an image as previously discussed herein.

In further embodiments, the medical imaging system 900 may include a computer system that enables a user to directly input an instruction to start or stop radiation emission and/or a position/orientation of the C-arm 904 or an imaging plan. In response, the computer system of the medical imaging system 900 causes radiation source 916 to start or stop radiation emission and causes the drive system 920 to move the C-arm 904 based on the input location or based on the input imaging plan.

When the patient 606 is undergoing certain surgical procedures, such as catheter-based cardiac interventions, X-ray fluoroscopy may be used to visualize a surgical instrument, e.g., a catheter, in real time as the surgical instrument (e.g., the catheter) travels throughout the patient 606. In some embodiments, during this type of procedure, the patient side cart 802 can be omitted as a single robotic arm 806 may be mounted to the patient table 602.

By way of example, a robotic arm 806 used during a catheter-based cardiac intervention deploys a catheter as a surgical tool 808. In these interventions, the medical imaging system 900 outputs a real time image to the display 820 via the computer system 604 as previously discussed herein. In some embodiments, a second medical imaging system 900 (e.g., a 3D ultrasound) may provide a real time 3D model of an anatomy of interest. In these embodiments the computer system 604 may register the 3D model to a fluoroscopic image, overlay the 3D model on the fluoroscopic image, and output the image to the display 820.

Similarly, when the patient 606 is undergoing ETON, X-ray fluoroscopy may be used to visualize an internal anatomy in real time. During this procedure, the medical imaging system 900 outputs a real time image to the display 820 via the computer system 604 as previously discussed herein.

While the operating room 600 is depicted as including the medical imaging system 900, in some embodiments the medical imaging system 900 may be omitted. For example, the patient 606 may undergo a surgical procedure wherein the medical imaging system 900 is not needed (e.g., when the patient 606 is undergoing a surgical procedure to remove a tumor). Furthermore, while FIG. 6 depicts the operating room 600 as including the computer system 604, in other embodiments the computer system 604 may be remote from the operating room 400 (e.g., in a different room of a hospital). Providing the computer system 604 in a different room than the operating room 400 allows the computer system 604 to be placed in a nonsterile environment. In some embodiments, the computer system 604 may be a node of a cloud computing environment.

In one embodiment, the computer system 604 may be a user computer system that is connected to a metaverse server. In this embodiment, a metaverse server may generate a metaverse that depicts the operating room 600. The metaverse server may generate a representation of the robotic surgical system 800, the medical imaging system 900, and the patient 606 as the patient is undergoing a surgical procedure. The metaverse server may update a position/orientation of the robotic surgical system 800 and the medical imaging system 900 within the metaverse as the operation is carried out. Furthermore, the metaverse server may populate a live video feed from the camera assembly 810 or an optical camera 616 (that is disposed within the operating room 600) into the metaverse. Furthermore, the metaverse server may populate an image captured by the medical imaging system, a preoperative image, and/or a 3D model overlaid on an image captured by the camera assembly 810 as previously discussed herein into the metaverse. In some embodiments, the metaverse server outputs the metaverse to a display within a VR headset.

During a surgical procedure, the position of the tools 808 may be tracked by various systems and methods. Some examples of such suitable systems and methods are disclosed in WO 2021/087027 and WO 2021/011760 each of which is incorporated herein by reference in their entirety. The computer systems (e.g., a metaverse server) may use the tracked positions to augment a surgeon's ability to perform a surgical procedure. In one embodiment, a metaverse server populates the surgical tools 808 into a metaverse based on the tracked positions.

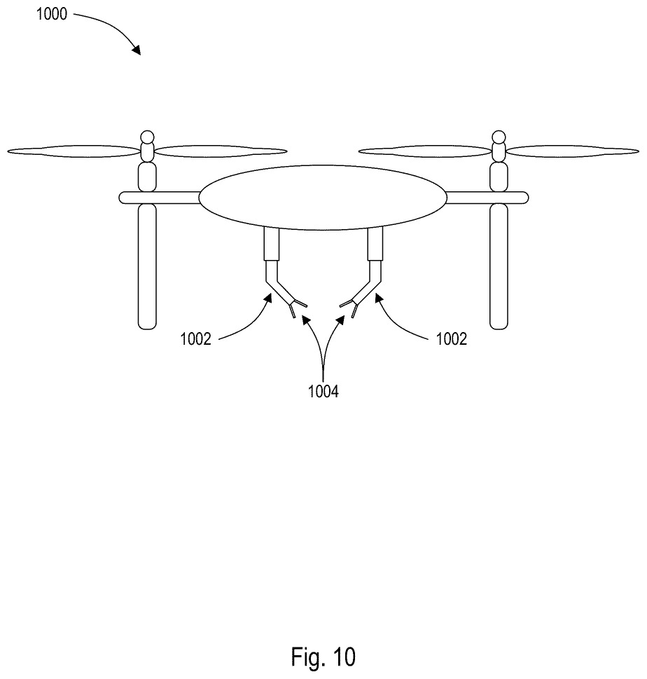

Referring now to FIG. 10 an autonomous vehicle 1000 is shown in accordance with an exemplary embodiment. While the autonomous vehicle 1000 is depicted and referred to as a drone, it is understood that the autonomous vehicle may be any type of autonomous vehicle. When not in use, the drone 1000 may be stored in a storage room of a facility (e.g., a hospital). The storage room may include docking elements for charging the battery of the drone 1000.

The drone 1000 includes robotic arms 1002 each having a plurality of robotic fingers 1004. The robotic arms 1002 are connected to the body of the drone 1000 and proximal ends of the fingers 1004 are connected to a distal end of a robotic arm 1002. While the robotic arms 1002 are depicted as vertically below the body of the drone 1000, in other embodiments, the robotic arms 1002 are attached to the body of the drone 1000 at a different location. The battery of the drone 1000 powers the robotic arms 1002 and the robotic fingers 1004. While FIG. 10 depicts the drone 1000 as including two robotic arms 1002, in other embodiments, the drone 1000 may have more or less robotic arms 1002 (e.g., 1, 3, 4, etc.).

The robotic arm 1002 and the robotic fingers 1004 are articulable and therefore moveable between a plurality of positions. More specifically, the robotic fingers 1004 are moveable between a fully open and a fully closed position and any number of positions therebetween. Furthermore, the robotic fingers 1004 are rotatable 360° in a clockwise and counterclockwise direction.

In one embodiment, the autonomous vehicle 1000 is configured to remove a surgical tool 808 from and attach a surgical tool 808 to a robotic arm 806 of the robotic surgical system 800. While the autonomous vehicle 1000 is depicted and referred to as a drone, it is understood that the autonomous vehicle may be any type of autonomous vehicle capable of carrying out the various actions discussed herein.

The robotic fingers 1004 are configured to remove a surgical tool 808 from a robotic arm 806. In one example, wherein a surgical tool 808 is attached to the robotic arm 806 via a threaded attachment, the robotic fingers 1004 move from an open position to a closed position. In the closed position, the robotic fingers grip the surgical tool 808. After gripping the surgical tool 808, the robotic fingers 1004 rotate to remove the surgical tool 808 from the robotic arm 806. In another example, wherein a surgical tool 808 is attached to the robotic arm 806 via a tongue and groove attachment interface or a snap fit attachment interface, the robotic fingers 1004 move from an open position to a closed position. In the closed position, the robotic fingers grip the surgical tool 808 at the attachment interface. When in the closed position, the robotic fingers 1004 supply sufficient force to cause the surgical tool 808 to disengage from the robotic arm 806. Furthermore, after removing a surgical tool 808 from the robotic surgical system 800, the robotic fingers 1004 may continue to grip the removed surgical tool 808 and carry the surgical tool 808 while the drone 1000 is in flight.

A user of an external computer system that is connected to the computer system of the drone 1000 may input a target destination (e.g., coordinate position, operating room, etc.) and a surgical tool 808 to remove from the robotic surgical system 800 and/or a surgical tool 808 to add (e.g., replace a removed tool) to the robotic surgical system 800 which causes the external computer system to send a signal indicative of the input to the computer system of the drone 1000. In response to receiving this signal, the computer system of the drone 1000 causes the drone 1000 to decouple from the docking elements and travel to the target destination. In some embodiments, wherein the input includes a surgical tool 808 to add to the robotic surgical system 800, the drone 1000 may obtain the desired surgical tool 808 from storage via the robotic fingers 1004 and carry the surgical tool 808 to the target destination. Since the drone 1000 is an AV, the drone 1000 can automatically travel to the target destination and may automatically obtain the desired surgical tool 808. In another embodiment, a user of the external computer system may manually pilot the drone 1000 to obtain the desired surgical tool 808 and may pilot the drone 1000 to the target destination.

Upon arriving at the target destination, the drone 1000 positions itself to remove or add the desired surgical tool 808 based on the input. In some embodiments, an optical camera(s) of the drone 1000 may automatically capture optical images of the surgical tools 808 and sends the images to a computer system (e.g., the computer systems of the drones, nodes of a cloud computing system etc.). In response to receiving the images, the computer system may employ surgical tool recognition software that automatically identifies surgical tool 808 to be removed and/or a robotic arm 806 to add a surgical tool 808 to the received optical images and sends position signals to the computer system of the drone 1000 indicative thereof. In response to receiving these signals, the computer system of the drone 1000 causes the drone 1000 to maneuver to a position to remove and/or add a surgical tool 808 to a robotic arm 806. In other embodiments, a user of the external computer system pilots the drone 1000 to a position to remove and/or add a surgical tool 808 to a robotic arm 806.

Once in a proper position/orientation, the drone 1000 may automatically remove and/or add a surgical tool 808 to a robotic arm 806. In some embodiments, the drone 1000 may remove a first surgical tool 808 from a robotic arm 806 and replace the surgical tool 808 with a different second surgical tool 808. In another embodiment, a user of the external computer system may pilot the drone to remove and/or add a surgical tool 808 to a robotic arm 806. When the drone 1000 has finished removing and/or adding the surgical tool, the drone 1000 may return to the storage room automatically or via a human pilot. If the drone 1000 has removed a surgical tool 808, the drone 1000 may carry the surgical tool to storage.

As discussed with respect to the exemplary drone 500, in some embodiments the drone 1000 is connected to an external computer system and includes an optical camera. In one embodiment, the external computer system may be a user computer system that is connected to a metaverse server. Stated another way, the drone 1000 may be connected to a user computer system 302 that is connected to a metaverse server 306. In this embodiment, a metaverse server may generate a metaverse that depicts the drone 1000. The metaverse server may update a position of the drone 1000 within the metaverse as it moves to the robotic surgical system 800. Once the drone 1000 arrives at the robotic surgical system 800 the metaverse server may populate an avatar representative of the robotic surgical system 800 into the metaverse, may update a position of the drone 1000, and may update a progress report of surgical tool 808 addition and/or removal. Once the surgical tools 808 have been added to and/or removed from the robotic surgical system 800, the metaverse server may update a position of the drone 1000 within the metaverse as it returns to a storage room. Furthermore, the metaverse server may populate a live video feed from the optical camera of the drone 1000 into the metaverse.

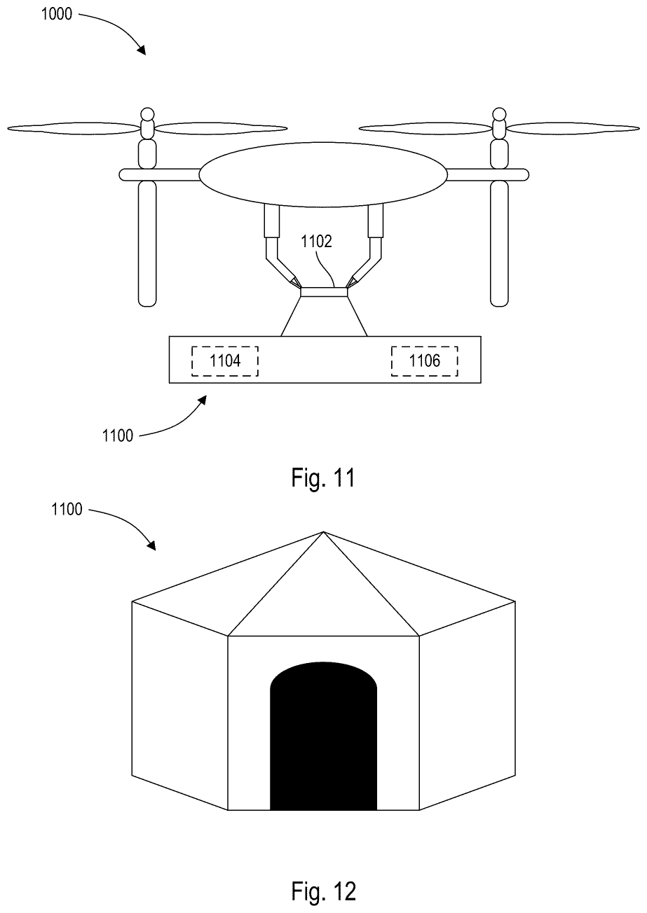

Referring now to FIG. 11, in another embodiment, the drone (or other autonomous vehicle) 1000 is configured to carry a tent 1100 in an undeployed position. As will be discussed in further detail herein, when deployed, the tent 1100 provides a sterile environment for carrying out various medical procedures including, but not limited to, a surgical procedure and/or a medical imaging procedure.

As depicted in FIG. 11, the drone 1000 grips a support bar 1102 that is connected to the tent 1100 when the robotic fingers 1004 are in a closed position. Upon moving the robotic fingers 1004 to an open position, the drone 1000 releases the tent 1100. In some embodiments, the tent 1100 and a pump 1104 that is connected to and in communication with a computer system 1106. The computer system 1106 is connected to and in communication with the computer system of the drone 1000. After the drone 1000 releases the tent 1100, the computer system of the drone 1000 sends a signal to the computer system 1106 to deploy the tent 1100. Upon receiving this signal, the computer system 1106 activates the pump 1104 which causes the tent 1100 to deploy (FIG. 12). When deployed, the pump 1104 may remain active such that the interior of the tent 1100 has a negative pressure.

A user of an external computer system that is connected to the computer system of the drone 1000 may input a target destination (e.g., coordinate position) which causes the external computer system to send a signal indicative of the input to the computer system of the drone 1000. In response to receiving this signal, the computer system of the drone 1000 causes the drone 1000 to decouple from the docking elements. In some embodiments, the drone 1000 may obtain the tent 1100 from storage via the robotic fingers 1004 and carry the tent 1100 to the target destination. Since the drone 1000 is an AV, the drone 1000 can automatically obtain the tent 1100 and can automatically travel to the target destination. In another embodiment, a user of the external computer system may manually pilot the drone 1000 to obtain the tent 1100 and may pilot the drone 1000 to the target destination.

Upon arriving at the target destination, the drone 1000 positions itself to release the tent 1100. In some embodiments, an optical camera(s) of the drone 1000 may automatically capture optical images of the target destination and sends the images to a computer system (e.g., the computer systems of the drones, nodes of a cloud computing system etc.). In response to receiving the images, the computer system may employ optical image recognition software that automatically identifies the target destination within the received optical images and sends position signals to the computer system of the drone 1000 indicative thereof. In response to receiving these signals, the computer system of the drone 1000 causes the drone 1000 to maneuver to a position/orientation indicated by those signals. In other embodiments, a user of the external computer system pilots the drone 1000 to a position to release the tent 1100.

Once in a proper position/orientation, the drone 1000 may automatically release the tent 1100. In another embodiment, a user of the external computer system may pilot the drone to release the tent 1100. When the drone 1000 has finished releasing the tent 1100, the drone 1000 may return to the storage room automatically or via a human pilot.

As discussed with respect to the exemplary drone 500, in some embodiments the drone 1000 is connected to an external computer system and includes an optical camera. In one embodiment, the external computer system may be a user computer system that is connected to a metaverse server. Stated another way, the drone 1000 may be connected to a user computer system 302 that is connected to a metaverse server 306. In this embodiment, a metaverse server may generate a metaverse that depicts the drone 1000. The metaverse server may update a position of the drone 1000 within the metaverse as it moves to target destination. Once the drone 1000 arrives at the target destination the metaverse server may populate a graphical representation of the target destination into the metaverse, may update a position of the drone 1000, and may update a progress of tent deployment. Once the tent 1100 has been deployed, the metaverse server may update a position of the drone 1000 within the metaverse as it returns to a storage room. Furthermore, the metaverse server may populate a live video feed from the optical camera of the drone 1000 into the metaverse.

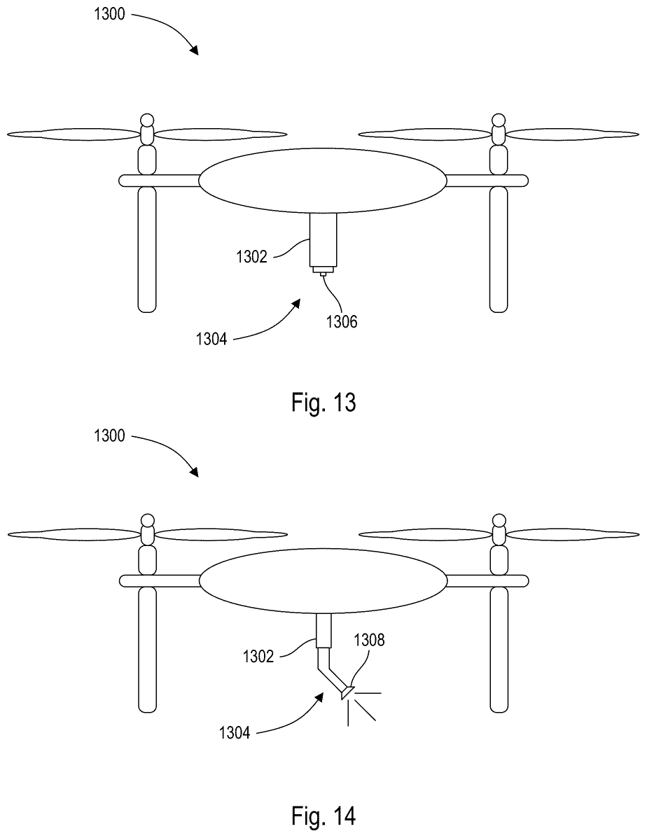

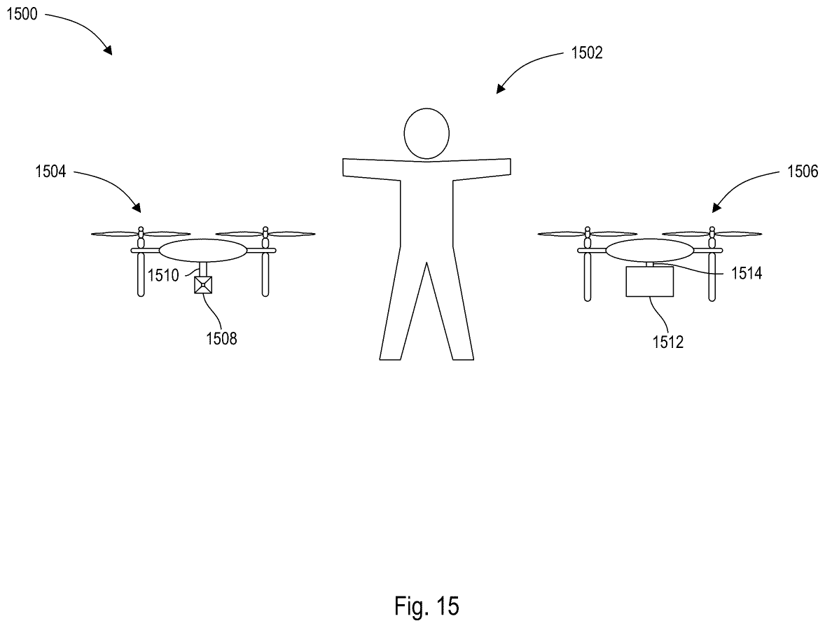

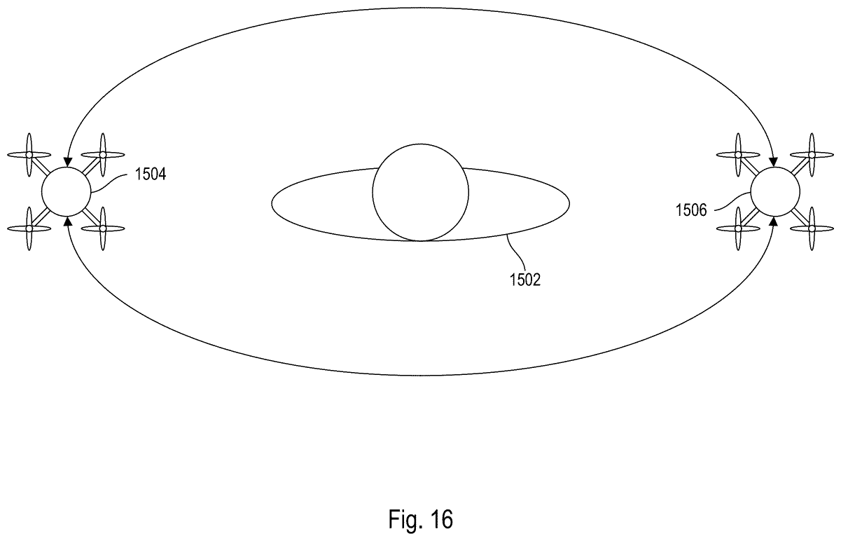

Referring now to FIGS. 13 and 14, an autonomous vehicle 1300 is shown in accordance with an exemplary embodiment. In this embodiment, the autonomous vehicle 1300 is configured to sterilize an environment (e.g., the operating room 600, the interior of the tent 1200, etc.). While the autonomous vehicle 1300 is depicted and referred to as a drone, it is understood that the autonomous vehicle may be any type of autonomous vehicle. When not in use, the drone 1300 may be stored in a storage room of a facility (e.g., a hospital). The storage room may include docking elements for charging the battery of the drone 1300.

The done 1300 includes a robotic arm 1302 with a sterilization element 1304 connected thereto. The robotic arm 1302 is connected to the body of the drone 1300 and proximal ends of the sterilization element 1304 is connected to a distal end of the robotic arm 1302. While the robotic arm 1302 is depicted as being positioned vertically below the body of the drone 1300, in other embodiments, the robotic arm 1302 is attached to the body of the drone 1300 at a different location. The battery of the drone 1300 powers the robotic arm 1302 and the robotic sterilization element 1304. The robotic arm 1302 and the sterilization element 1304 are articulable and therefore moveable between a plurality of positions. While FIGS. 13 and 14 show the drone 1300 including one robotic arm 1302 with one sterilization element 1304, in other embodiments, the drone 1300 may include more than one robotic arm 1302 each connected to a different sterilization element 1304.

Referring now to FIG. 13, in this embodiment, the sterilization element 1304 includes an aerosol spray cannister 1306 carrying a disinfecting solution (e.g., including isopropyl alcohol) capable of sterilizing an environment. Referring now to FIG. 14, the sterilization element 1304 includes a light source 1308 (e.g., an ultraviolent light source) that is also capable of sterilizing an environment.

Upon arriving at a target destination, (e.g., the operating room 600 or the tent 1200), the computer system of the drone 1300 causes the sterilization element 1304 to begin a sterilization procedure (e.g., causes the spray cannister 1306 to emit the disinfecting solution and/or causes the light source 1308 to emit ultraviolet radiation). When the sterilization procedure is complete, that is when the drone 1300 has completely sterilized the environment of the target destination, the drone 1300 may return to storage.

A user of an external computer system that is connected to the computer system of the drone 1300 may input a target destination (e.g., coordinate position) which causes the external computer system to send a signal indicative of the input to the computer system of the drone 1300. In response to receiving this signal, the computer system of the drone 1300 causes the drone 1300 to decouple from the docking elements. Since the drone 1300 is an AV, the drone 1300 can automatically travel to the target destination. In another embodiment, a user of the external computer system may manually pilot the drone 1300 to the target destination.

Upon arriving at the target destination, the drone 1300 positions itself to sterilize the target destination. In some embodiments, an optical camera(s) of the drone 1300 may automatically capture optical images of the target destination and send the images to a computer system (e.g., the computer systems of the drones, nodes of a cloud computing system etc.). In response to receiving the images, the computer system may employ optical image recognition software that automatically identifies the target destination within the received optical images and sends position/orientation signals to the computer system of the drone 1300 indicative thereof. In response to receiving these signals, the computer system of the drone 1300 causes the drone 1300 to maneuver to a desired position/orientation. In other embodiments, a user of the external computer system pilots the drone 1300.

Once in a proper position, the drone 1300 may automatically begin a sterilization procedure. In another embodiment, a user of the external computer system may pilot the drone to sterilize an environment. When the drone 1300 has finished sterilizing the environment, the drone 1300 may return to the storage room automatically or via a human pilot.

As discussed with respect to the exemplary drone 500, in some embodiments the drone 1300 is connected to an external computer system and includes an optical camera. In one embodiment, the external computer system may be a user computer system that is connected to a metaverse server. Stated another way, the drone 1300 may be connected to a user computer system that is connected to a metaverse server. In this embodiment, a metaverse server may generate a metaverse that depicts the drone 1300. The metaverse server may update a position of the drone 1300 within the metaverse as it moves to a target destination. Once the drone 1300 arrives at the target destination the metaverse server may populate a graphical representation of the target destination into the metaverse, may update a position of the drone 1300, and may update a progress sterilization. Once the environment has been sterilized, the metaverse server may update a position of the drone 1300 within the metaverse as it returns to a storage room. Furthermore, the metaverse server may populate a live video feed from the optical camera of the drone 1300 into the metaverse.

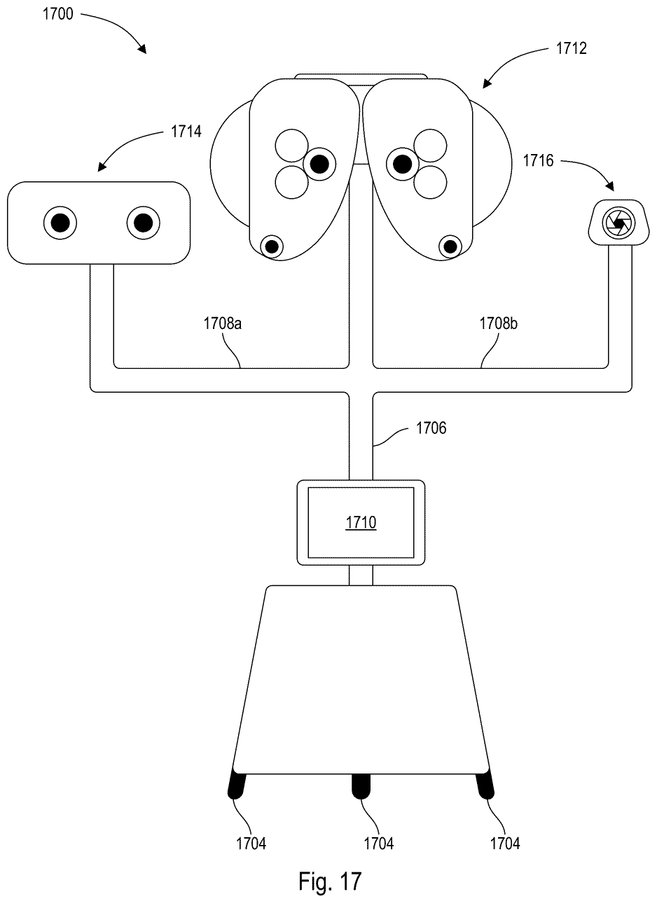

Referring now to FIG. 17, an optometric robot 1700 is shown in accordance with an exemplary embodiment. The optometric robot is an AV and as such, the AV 1700 includes wheels 1702, a drive system, sensors, and a computer system needed to autonomously pilot the optometric robot 1700. The optometric robot 1700 may pilot itself from a storage room to an exam room or other location (e.g., a patient's home) based on a predetermined schedule (e.g., an exam schedule) or based on a user input, e.g., transmitted to the robot via a remote-control station. When an exam is complete and the optometric robot 1700 is no longer needed, the optometric robot 1700 may automatically return to the storage room and may automatically connect to docking elements disposed therein.

The optometric robot 1700 includes a housing 1704 that is connected to the wheels 1702. The housing 1704 includes various electronic components (e.g., computer system., sensors, drive system, etc.) needed to operate the optometric robot 1700. The optometric robot 1700 further includes a vertical support arm 1706 connected to and extending perpendicular from the housing 1704. The vertical support arm 1706 is configured to move vertically with respect to the housing 1704. Accordingly, the vertical support arm 1706 is configured to vertically move devices connected thereto. The optometric robot 1700 also includes horizontal support arms 1708a and 1708b that re connected to and extend perpendicular from the vertical support arm 1706. As such, the vertical support arm 1706 is configured to move the horizontal support arms 1708.

The optometric robot 1700 further includes a display (e.g., a tablet) 1710. The tablet includes or is connected to the computer system of the optometric robot 1700. The display 1710 also includes an optical camera, a speaker, and a microphone (not shown) that allow a patient to establish a video conference session with a medical professional (e.g., an optometrist) during an exam.

The optometric robot 1700 includes various elements for carrying out an eye exam including a phoropter 1712, an autorefractor 1714, and a fundus camera 1716. The phoropter 1712 is connected to vertical support arm 1706, the autorefractor 1714 is connected to the horizontal support arm 1708a, and the fundus camera 1716 is connected to the horizontal support arm 1708b. The phoropter 1712, the autorefractor 1714 and the fundus camera 1716 are connected to and in communication with the computer system of the optometric robot 1700.

The computer system of the optometric robot 1700 is connected to and in communication with an external computer system. In some embodiments, a user of the external computer system may input a target destination (e.g., coordinate position, address, exam room location, etc.) which causes the external computer system to send a signal indicative of the input to the computer system of the optometric robot 1700. In response to receiving this signal, the computer system of the optometric robot 1700 causes the optometric robot 1700 to decouple from the docking elements and travel to the target destination. Since the optometric robot 1700 is an AV, the optometric robot 1700 can automatically travel to the target destination. In another embodiment, a user of the external computer system may manually pilot the optometric robot 1700 to the target destination.

Upon arriving at the target destination, the optometric robot 1700 positions itself relative to a patient. In some embodiments, an optical camera(s) of the optometric robot 1700 may automatically capture optical images of the target destination/patient and send the images to a computer system (e.g., the computer systems of the optometric robot 1700, nodes of a cloud computing system etc.). In response to receiving the images, the computer system may employ optical image recognition software that automatically identifies the target destination/patient within the received optical images and sends position/orientation signals to the computer system of the optometric robot 1700 indicative thereof. In response to receiving these signals, the computer system of the optometric robot 1700 causes the optometric robot 1700 to maneuver to a desired position/orientation and causes the vertical support arm to align at least one of the phoropter 1712, the autorefractor 1714, or the fundus camera 1716 with the eyes of a patient.

Once at least one of the phoropter 1712, the autorefractor 1714, or the fundus camera 1716 are aligned with the patient, a user (e.g., an optometrist, ophthalmologist, etc.) of an external computer system may begin an eye exam via video conferencing using the display 1710 to communicate with the patient. In some embodiments, the user of the computer system may cause the autorefractor 1714 to align with the patient. When properly aligned, the user of the computer system may employ the autorefractor 1714 to determine a lens prescription for the patient. After the lens prescription is determined, the computer system of the optometric robot 1700 may automatically change lenses of the phoropter 1712 to corresponding lenses. When adjusted the, the user of the computer system may cause phoropter 1712 to align with the eyes of the patient. The user of the external computer system may verify the lens prescription for the patient by inputting a lens prescription for the patient into the external computer system which causes the external computer system to send a corresponding signal to the computer system of the optometric robot 1700. In response to receiving this signal, the computer system of the optometric robot 1700 causes the phoropter 1712 to change lenses of the phoropter 1712 based on the input. The user of the external computer system is able to speak with the patient via video conferencing to verify the lens prescription. Before or after determining the lens prescription for the patient, the user of the external computer system may cause the fundus camera 1716 to align with a left or right eye of the patient. Once aligned, the user may photograph the fundus. The computer system of the optometric robot 1700 then sends the image to the external computer system for viewing by the user. This process is repeated for the opposite eye. This allows a user of the external computer system to diagnose various ailments (e.g., diabetes, age-macular degeneration (AMD), glaucoma, multiple sclerosis, neoplasm, etc.).

While the above describes the optometric robot 1700 as including the phoropter 1712, the autorefractor 1714, and the fundus camera 1716, it is understood that other devices for performing an eye exam (e.g., tonometer, vision screener, digital Snellen chart, etc.) may be included in the optometric robot 1700 by replacing at least one of the phoropter 1712, the autorefractor 1714, or the fundus camera 1716 or by providing an optometric robot 1700 with additional arms that support additional devices.

In one embodiment, the external computer system that is connected to the computer system of the optometric robot 1700 is connected to a metaverse server. Stated another way, the optometric robot 1700 may be connected to a user computer system 302 that is connected to a metaverse server 306. In this embodiment, a metaverse server may generate a metaverse that depicts the optometric robot 1700. The metaverse server may update a position of optometric robot 1700 within the metaverse as it moves to target destination. Once the optometric robot 1700 arrives at the target destination the metaverse server may populate a graphical representation of the target destination and an avatar corresponding to the patient into the metaverse, may update a position of the optometric robot 1700, and may update a progress of the eye exam. Once the exam is complete, the metaverse server may update a position of the optometric robot 1700 within the metaverse as it returns to a storage room. Furthermore, the metaverse server may populate a live video feed from the optical camera of the optometric robot 1700 into the metaverse.

While the robotic optometric robot 1700 is described as an AV, in some embodiments, the AV may be a drone. In these embodiments, a drone (e.g., the drone 500) carries or includes the components of the elements of the patient optometric robot 1700 needed to perform an eye exam (e.g., the phoropter 1712, the autorefractor 1714, and the fundus camera 1716).

As previously discussed, the above may be implemented by way of computer readable instructions, encoded or embedded on computer readable storage medium (which excludes transitory medium), which, when executed by a processor(s), cause the processor(s) to carry out various methods relating to the present disclosure.

While the invention has been illustrated and described in detail in the drawings and foregoing description, such illustration and description are to be considered illustrative or exemplary and not restrictive; embodiments of the present disclosure are not limited to the disclosed embodiments. Other variations to the disclosed embodiments can be understood and effected by those skilled in the art in practicing embodiments of the present disclosure, from a study of the drawings, the disclosure, and the appended claims.

In the claims, the word “comprising” does not exclude other elements or steps, and the indefinite article “a” or “an” does not exclude a plurality. A single processor or other processing unit may fulfill the functions of several items recited in the claims. The mere fact that certain measures are recited in mutually different dependent claims does not indicate that a combination of these measured cannot be used to advantage. Any reference signs in the claims should not be construed as limiting the scope.

1. A system comprising:
a robotic surgical system that includes a surgical tool; and
an autonomous vehicle configured to remove the surgical tool from the robotic surgical system.

2. The system of claim 1, wherein the autonomous vehicle is further configured to connect to a metaverse.

3. The system of claim 1, wherein the robotic surgical system is configured to connect to a metaverse.

4. The system of claim 1, wherein the surgical tool is a first surgical tool and the autonomous vehicle is further configured to attach a second surgical tool to the robotic surgical system.

5. The system of claim 1, wherein the robotic surgical system further includes a robotic arm and the surgical tool is removably attached to the robotic arm.

6. The system of claim 5, wherein the robotic arm is a first robotic arm, and the surgical tool is a first surgical tool and the robotic surgical system further includes:
a second robotic arm, and
a second surgical tool attached to the second robotic arm, wherein the autonomous vehicle is configured to remove the second surgical tool from the second robotic arm.

7. The surgical system of claim 6, wherein the autonomous vehicle is configured to attach a third surgical tool to the second robotic arm.

8. The system of claim 1, wherein the metaverse includes a real time position of the autonomous vehicle.

9. The system of claim 1, wherein the autonomous vehicle includes an optical camera and the
metaverse includes a real time video provided by the optical camera.

10. The system of claim 1, wherein the autonomous vehicle is a drone.

11. The system of claim 10, wherein the drone is configured to automatically remove the surgical tool.

12. The system of claim 1, wherein the robotic surgical system is an autonomous vehicle.

13. The system if claim 1, further comprising:
a metaverse; and
a user computer system,
wherein the user computer system and the autonomous vehicle are connected to the metaverse, and
wherein the user computer system is configured to pilot the autonomous vehicle.

14. The system of claim 1, further comprising:
a metaverse.
a medical imaging system configured to image an internal anatomy of a subject and output the image to the metaverse.

15. The system of claim 14, wherein the output image is a real time image.

16. A system comprising:
a first autonomous vehicle and a second autonomous vehicle configured to image an internal anatomy of a subject and further configured to connect to a metaverse.

17. The system of claim 16, wherein the first autonomous vehicle includes a radiation source that is configured to emit radiation that is attenuated by the subject and the second autonomous vehicle includes a radiation detector configured to detect the attenuated radiation.

18. The system of claim 16, wherein the first autonomous vehicle and the second autonomous vehicle are configured to automatically image the subject.

19. The system of claim 16, wherein the metaverse includes a real time position of the first autonomous vehicle and the second autonomous vehicle.

20. The system of claim 16, wherein the first autonomous vehicle and the second autonomous vehicle are drones.

21-36. canceled
